(19) United States
(12) Patent Application Publication
(10) Pub. No.: US 2026/0363920 A1
(43) Pub. Date: Nov. 19, 2026

(54) DEFECATION PAD INCLUDING TRIPLE BACKFLOW-PREVENTING FILTERS AND ANTIBACTERIAL ABSORBENT BODIES

(71) Applicant: EUN JEA SUNG, Goyang-si (KR)

(72) Inventor: EUN JEA SUNG, Goyang-si (KR)

(21) Appl. No.: 19/710,497

(22) Filed: Jun. 17, 2026

Related U.S. Application Data
(63) Continuation of application No. PCT/KR2026/003862, filed on Mar. 10, 2026.

(30) Foreign Application Priority Data
May 16, 2025 (KR) 10-2025-0064113

(51) Int. Cl. A61F 13/51; A61F 13/15

(52) U.S. Cl. CPC A61F 13/51; A61F 13/15577; A61F 2013/1513; A61F 2013/15991; A61F 2013/51047

(57) ABSTRACT

The present invention relates to a defecation pad (10) configured by an ordered combination of a plurality of layers, and the plurality of layers include: a first filter layer (12); a first antibacterial absorbent body layer (13) onto which super absorbent polymer particles are applied for absorption/storage of urine; a second filter layer (14); a second antibacterial absorbent body layer (15) onto which super absorbent polymer particles are applied for the absorption/storage of the urine; a cloud sheet (16) formed of a woven fabric having a net structure for aligning particles in contact with an upper portion and a lower portion so as to be uniformly distributed; a third antibacterial absorbent body layer (17) onto which super absorbent polymer particles are applied for the absorption/storage of the urine; and a third filter layer (18).

### CROSS-REFERENCE TO RELATED APPLICATIONS

This application is a continuation of PCT Patent Application No. PCT/KR2026/003862, filed on Mar. 10, 2026, which claims the benefit of Korean Patent Application No. KR 10-2025-0064113, filed on May 16, 2025.

### TECHNICAL FIELD

The present invention relates to the field of sanitary pads, and more particularly to a configuration of a defecation pad including triple filters and antibacterial absorbent bodies so as to achieve rapid absorption of urine and prevention of backflow of odor and bacteria.

### BACKGROUND ART

A defecation pad is configured as a multilayer structure including a top sheet, an absorbent layer, and a bottom waterproof layer, and is designed to rapidly absorb the excrement of a companion animal. The top sheet is mainly formed of a nonwoven fabric or a mesh material so as to allow urine to permeate downward, but a nonwoven fabric material has a problem in that the urine remains on the surface and wets the paw pads. In addition, although the absorbent layer is a single super absorbent polymer (SAP) absorbent body capable of absorbing a large amount of urine, an antibacterial action is not performed immediately upon absorption, and therefore bacterial proliferation and odor persist when the pad is used for a long period of time. Although the bottom waterproof layer, made of a polyethylene (PE) film, prevents external leakage, its backflow-preventing function is insufficient, and therefore leakage or backflow may occur when the absorption capacity is exceeded.

In the prior art, a method of increasing the weight or thickness of the absorbent body has been used in order to improve the absorption rate and absorption efficiency, but this increases the product thickness and raises the manufacturing cost. In addition, even though the structure is a multilayer structure, a separate filter layer for preventing backflow is absent, and therefore the urine is not isolated after a certain amount of urine has been absorbed, so that a backflow occurs, and complaints have been raised in terms of user convenience and sanitary management.

As described above, since a conventional defecation pad focuses only on the absorption amount and the waterproof function and fails to provide a comprehensive solution for securing backflow prevention and antibacterial properties, a defecation pad having a new structure capable of simultaneously improving the companion animal sanitary maintenance and the user convenience is required.

Korean Intellectual Property Office Laid-Open Patent Publication No. 10-2023-0086884 discloses a “defecation pad.”

### DISCLOSURE

### Technical Problem

The present invention has been devised in response to the above-described background art, and the problem to be solved by the present invention is to provide a solution for the problem that, in the defecation pad according to the background art, urine remains on the top sheet so that the paw pads of a companion animal become wet, for the problem that the absorbent layer fails to perform an antibacterial action immediately so that, when the pad is used for a long period of time, bacterial proliferation and odor caused by secondary bacterial proliferation persist, and for paw-print (a phenomenon in which the wetted paw pads of a companion animal leave marks on a surrounding floor) and odor backflow caused by an insufficient backflow-preventing function.

Meanwhile, the technical problems to be achieved by the present invention are not limited to the above-mentioned technical problems, and various technical problems may be included within a scope that is obvious to a person of ordinary skill in the art from the contents described below.

### Technical Solution

In order to solve the above problem, the following means is described.

A defecation pad according to the present invention is formed by a plurality of layers being combined in order, and the plurality of layers include: a first filter layer (a bamboo charcoal filter layer); a first antibacterial absorbent body layer onto which super absorbent polymer particles are applied for absorption and storage of urine; a second filter layer (a first permeation control filter); a second antibacterial absorbent body layer onto which super absorbent polymer particles are applied for absorption and storage of urine; a cloud sheet formed of a woven fabric having a net structure for aligning the particles in contact with an upper portion and a lower portion so as to be uniformly distributed; a third antibacterial absorbent body layer onto which super absorbent polymer particles are applied for absorption and storage of urine; and a third filter layer (a second permeation control filter).

In addition, the plurality of layers may further include, at an uppermost end, a water-repellent top sheet layer in which drainage holes are arranged, and the top sheet layer is formed of various materials such as polyethylene (PE) mesh, a nonwoven fabric, or Korean paper (hanji).

In addition, the plurality of layers further include, at a lowermost end, a waterproof bottom sheet layer which increases a frictional force with the ground and prevents an outflow of the urine.

The first filter layer includes: a first absorbent surface formed of black bamboo charcoal for concealing marks of the urine and for an antibacterial action; a second absorbent surface having a higher liquid absorption resistance than the first absorbent surface; and a third absorbent surface having a still higher liquid absorption resistance than the second absorbent surface.

In at least one of the first to third antibacterial absorbent body layers, S-type super absorbent polymer particles having a fast absorption rate are arranged at an upper portion, and N-type super absorbent polymer particles having a large absorption amount are arranged at a lower portion, and the S-type super absorbent polymer particles and the N-type super absorbent polymer particles may have, for example, a weight ratio of 1:3, but a proportion of the N-type super absorbent polymer particles is higher when the defecation pad is for a large-breed dog than when the defecation pad is for a small-breed dog.

In addition, an antibacterial agent based on silver ions or copper ions is applied to the surfaces of the super absorbent polymer particles used in the first to third antibacterial absorbent body layers.

### Advantageous Effects

According to the present invention, through the multilayer structure in which the first to third filter layers, the first to third antibacterial absorbent body layers, the cloud sheet, and the top and bottom sheet layers are combined in order, rapid permeation of urine, multiple backflow prevention, antibacterial absorption, and a waterproof function can be integrally implemented.

By adopting a water-repellent structure in which drainage holes are arranged in the top sheet layer, the urine permeates downward immediately, so that the surface drying capability can be improved.

By forming the top sheet layer of at least one material among polyethylene (PE) mesh, a nonwoven fabric, and Korean paper (hanji), durability and cost competitiveness can be simultaneously secured.

Owing to the waterproof and anti-slip functions of the bottom sheet layer, external leakage can be completely blocked and the fixing force of the pad can be increased.

Through the triple absorbent surface arrangement of the first filter layer, urine marks can be concealed by the black surface, and efficient backflow prevention can be achieved by sequentially increasing the liquid resistance.

By arranging the S-type super absorbent polymer (SAP) at an upper portion and the N-type SAP at a lower portion in at least one of the first, second, and third antibacterial absorbent body layers, fast initial absorption and large-capacity storage can be simultaneously implemented.

Owing to the layer-by-layer SAP configuration of the first to third antibacterial absorbent body layers, a uniform absorption distribution can be implemented without local blocking even under supersaturation.

By setting the weight ratio of the S-type and N-type SAP particles to 1:3, an optimal balance between the absorption rate and the storage capacity can be achieved. However, although the weight ratio may be 1:3, when the defecation pad is for a large-breed dog, the proportion of the N-type super absorbent polymer particles may be made higher than when the defecation pad is for a small-breed dog, and an optimization effect in accordance with the size of the dog breed can thereby be produced.

By applying or injecting an antibacterial agent based on silver ions or copper ions onto the surfaces of the first to third antibacterial absorbent body layers, bacterial proliferation can be suppressed and odor can be reduced over a long period of time.

Meanwhile, the effects of the present disclosure are not limited to the above-mentioned effects, and various effects may be included within a scope that is obvious to a person of ordinary skill in the art from the contents described below.

### DESCRIPTION OF DRAWINGS

FIG. 1 shows an exemplary view and an exploded view of a defecation pad according to one embodiment of the present invention.

FIG. 2 shows an enlarged cross-sectional view illustrating a combined structure of a top sheet layer and a first filter layer according to one embodiment of the present invention.

FIG. 3 shows a plan view illustrating layer-by-layer arrangement of N-type and S-type super absorbent polymer particles disposed in a first antibacterial absorbent body layer according to one embodiment of the present invention.

FIG. 4 shows a perspective view schematically illustrating net-structured woven fabric of a cloud sheet according to one embodiment of the present invention.

FIG. 5 is an actual photograph of a defecation pad according to one embodiment of the present invention.

### BEST MODES OF THE INVENTION

Various embodiments are now described with reference to the drawings. In the present disclosure, various descriptions are presented in order to provide an understanding of the present disclosure. However, it is apparent that these embodiments may be practiced without these specific descriptions.

The term “or” is intended to mean an inclusive “or” rather than an exclusive “or.” In other words, unless otherwise specified or clear from the context, the expression “X uses A or B” is intended to mean one of the natural inclusive substitutions. In other words, when X uses A; X uses B; or X uses both A and B, the expression “X uses A or B” may be applied in any of these cases. In addition, the term “and/or” used in the present disclosure should be understood to refer to and include all possible combinations of one or more of the listed related items.

In addition, the terms “comprises” and/or “comprising” should be understood to mean that the corresponding feature and/or component is present. However, the terms “comprises” and/or “comprising” should be understood as not excluding the presence or addition of one or more other features, components, and/or groups thereof. In addition, unless otherwise specified or clear from the context that the singular form is indicated, the singular form in the present disclosure and in the claims should generally be construed to mean “one or more.”

In addition, the term “at least one of A or B” should be construed to mean “a case of comprising A only,” “a case of comprising B only,” and “a case combined into a configuration of A and B.”

The description of the presented embodiments is provided so that a person of ordinary skill in the art to which the present disclosure pertains may use or practice the present disclosure. Various modifications to these embodiments will be apparent to a person of ordinary skill in the art to which the present disclosure pertains. The general principles defined herein may be applied to other embodiments without departing from the scope of the present disclosure. Therefore, the present invention is not limited to the embodiments presented herein. The present invention is to be construed within the broadest scope consistent with the principles and novel features presented herein.

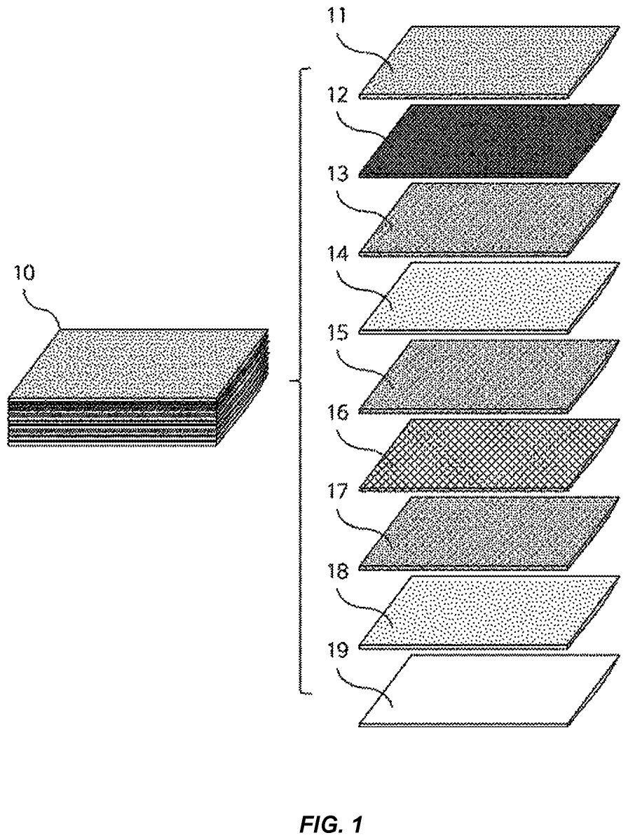

FIG. 1 shows an exemplary view and an exploded view of a defecation pad according to one embodiment of the present invention.

FIG. 1 is an exploded perspective view illustrating the overall configuration of a defecation pad 10 and a structure of each layer according to one embodiment of the present invention, and shows a multilayer defecation pad structure composed of a total of nine layers. The defecation pad 10 may be designed to comprehensively implement rapid urine permeation, multi-stage backflow prevention, antibacterial absorption, and a waterproof function.

A top sheet layer 11 may be disposed at the uppermost end. The top sheet layer 11 may be formed of various materials such as polyethylene (PE) mesh, a nonwoven fabric, Korean paper (hanji), or mulberry paper (dak paper), and may include a plurality of drainage holes for rapidly allowing urine to permeate. This structure can rapidly transfer urine to the lower layers and can prevent the urine from remaining on the surface, thereby preventing paw pads of a companion animal from becoming wet. In particular, when PE mesh is applied, the urine does not stay on the surface but moves to the lower layers, so that a fast drying effect can be implemented.

From the standpoint of a companion animal, since the urine does not remain on the uppermost layer that the paw pads contact after the companion animal has urinated on the defecation pad, the problem of the paw pads becoming wet can be solved. For example, even when a small-breed dog steps on the pad again and moves immediately after defecation, moisture is not transferred to the paws, so that indoor contamination can be prevented.

A first filter layer 12 may be disposed below the top sheet layer 11. The filter layer may be formed of a triple-structured functional nonwoven fabric consisting of a first absorbent surface that contains a bamboo charcoal component and exhibits a black color, a second absorbent surface having a higher liquid absorption resistance than the first absorbent surface, and a third absorbent surface having the highest resistance. This structure is capable of allowing urine to pass only in a downward direction and physically blocking upward backflow. In addition, the black surface may also perform a function of concealing urine marks. However, the first filter layer 12 is not necessarily limited to a structure for blocking backflow, and a material having excellent liquid absorption capability, a material having excellent waterproof properties, or a material having excellent water-repellent properties may be freely used as needed.

Here, the reason for including the bamboo charcoal component in the first absorbent surface is that the bamboo charcoal component can serve as a filter free of harmful substances and artificial additives, can assist an antibacterial action, and, owing to the dense permeability of the paper-type material, can produce an effect of high liquid absorption and, at the same time, prevention of SAP detachment.

A first antibacterial absorbent body layer 13 may be disposed therebelow. The absorbent body layer includes super absorbent polymer (SAP) particles, and S-type SAP, which has a small particle size and a fast absorption rate, may be arranged at an upper portion, while N-type SAP, which has a large storage capacity, may be stacked and arranged at a lower portion. The S-type SAP may denote a small SAP, and the N-type SAP may denote a normal SAP. Here, the S-type SAP may have a smaller particle size than the N-type SAP. In addition, the weight ratio of the two may be adjusted to 1:3. However, although the weight ratio may be 1:3, when the defecation pad is for a large-breed dog, the proportion of the N-type super absorbent polymer particles may be made higher than when the defecation pad is for a small-breed dog, and an optimization effect in accordance with the size of the dog breed can thereby be produced. In addition, an antibacterial agent based on silver ions or copper ions may be applied or injected onto the surfaces of the SAP, so that an antibacterial action can be initiated immediately upon urine absorption. The weight ratio of 1:3 is merely an example and is not limited thereto, but may be adjusted.

For example, even when a companion dog that is alone for a long time uses the pad for one day or longer, bacteria proliferation and odor generation can be suppressed, so that the sanitary condition can be stably maintained. This is a structure capable of overcoming the problem that odor begins to be generated within several hours in a general SAP-based pad.

Below the first antibacterial absorbent body layer 13, a second filter layer (the first permeation control filter, 14) and a second antibacterial absorbent body layer 15 may be sequentially disposed. The second filter layer 14 can serve as a one-way passage structure in the same manner as the first filter layer, and can block upward backflow through adjustment of the fiber density and the surface resistivity, and the like. The second antibacterial absorbent body layer 15 also includes SAP, and can additionally absorb urine that has not been processed in the first absorption, thereby increasing the storage capacity.

Such a repeated multilayer structure can extend the urine movement path and can induce the urine to be absorbed in a stepwise dispersed manner. For example, even when a large-breed dog discharges a large amount of urine, the urine is dispersed and stored downward before the upper absorbent layer becomes saturated, so that backflow can be prevented.

A cloud sheet 16 may be disposed below the second antibacterial absorbent body layer 15. The cloud sheet is configured as a net-shaped woven structure, and can uniformly fix the SAP particles on both the upper and lower surfaces. The fiber weaving density, the arrangement angle, and the like may be adjusted to induce the SAP to be aligned at regular intervals, and the cloud sheet may act so that liquid is diffused over the entire surface without being concentrated at a particular portion.

Similarly to the principle of ink spreading on paper, this can suppress local saturation (blow-out) and maximize the absorption efficiency of the entire SAP. In addition, the cloud sheet three-dimensionally fixes the SAP between the woven portions, so that the SAP can be prevented from being skewed or clumped even under the movement or pressure of a companion animal.

For example, even when a companion animal repeatedly urinates on a particular portion, the cloud sheet disperses the liquid so that the entire SAP operates effectively, thereby reducing local backflow and increasing the service life of the pad.

A third antibacterial absorbent body layer 17 and a third filter layer (the second permeation control filter, 18) may be disposed below the cloud sheet 16. The third antibacterial absorbent body layer 17 may be formed by stacking S-type SAP at an upper portion and N-type SAP at a lower portion at a weight ratio of 1:3, and may operate as a final storage stage within the pad.

This layer may be configured so that, after rapidly performing initial absorption of the urine that has been dispersed through the cloud sheet, the N-type SAP at the lower portion can stably store the urine for a long period of time.

The third filter layer 18 may be designed to reinforce a one-way passage function so that the urine is not re-drawn into the upper layers. This may be implemented by combining a fiber arrangement angle, a weaving density, a water-repellent treatment technique, and the like. In addition, the third filter layer is formed more densely than the second filter layer, thereby producing an effect of increasing the permeability so that the liquid is relatively evenly dispersed and absorbed upon liquid absorption.

At this time, the evenly dispersed liquid assists in preventing gel blocking.

For example, even when a companion animal sits on the pad for a long time, the urine in the lower layers is prevented from rising up to the upper portion and wetting the surface, so that a dry surface state can ultimately be maintained.

In addition, together with the cloud sheet, the third filter layer maintains a space and increases the distribution stability of the SAP, and prevents the backflow of urine and odor that are absorbed by and isolated within the SAP.

A bottom sheet layer 19 may be disposed at the lowermost end. The bottom sheet layer may be formed of a PE film or a waterproof synthetic resin corresponding thereto, and can completely block external urine leakage with a continuous film structure. The film thickness may be set to at least 0.02 mm in order to ensure durability and waterproof properties.

In addition, an anti-slip embossing pattern or a rubbery coating may be applied to an outer surface of the bottom sheet layer, and a fixing force can thereby be imparted so that the product is not easily pushed on the ground.

For example, since the pad can maintain a stable position without being pushed even when used on a slippery tile floor, sanitary use is made possible without being affected by the activity of a companion animal.

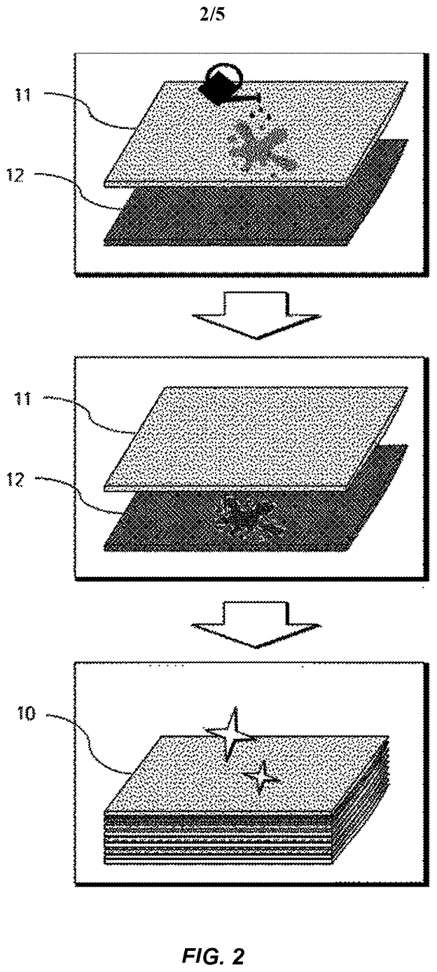

FIG. 2 shows an enlarged cross-sectional view illustrating a combined structure of a top sheet layer and a first filter layer according to one embodiment of the present invention.

The top sheet layer may be configured so that the urine can rapidly pass downward without staying on the surface even immediately after urination by a companion animal. In the present embodiment, the top sheet layer may be formed of PE mesh perforated with a plurality of drainage holes, and may alternatively be configured of a water-repellent-treated nonwoven fabric, or of Korean paper (hanji) and mulberry paper (dak paper) of a natural fiber type. Such a sheet may be configured of a single material, and may also be compositely formed in a double-sheet structure in which a PE mesh layer and a nonwoven fabric are stacked. Through such a structure, the top sheet layer can rapidly guide the urine downward and, at the same time, can suppress the scattering or surface residue of the urine, thereby maintaining a comfortable surface state.

The first filter layer may be combined below the top sheet layer in an adhesive manner or a heat-fusion manner. The first filter layer may be formed in a triple structure having directionality in which the liquid resistance gradually increases from the upper portion toward the lower portion. The first absorbent surface located at the top is configured of a low-density nonwoven fabric, and may be colored black so that, even after the urine has come into contact therewith, stain marks are not visible in terms of external appearance. In addition, adsorbent particles such as charcoal fibers or zeolite may be mixed into this absorbent surface so as to perform a deodorizing function.

The second absorbent surface located in the middle may be configured of a medium-density water-repellent-treated nonwoven fabric or an irregularly woven net fiber layer, and can induce the urine that has flowed in from the upper portion to move only downward, while suppressing upward backflow. The third absorbent surface located at the bottom may be configured of a high-density nonwoven fabric or a membrane film including micro-holes. This structure can fundamentally block the re-discharge of the urine that has passed downward, to the upper portion by external pressure or the like. For example, a silicone-based water-repellent-coated nonwoven fabric or a microporous polyurethane film may be applied, and such a multilayer resistance structure can reinforce one-way flow so as to induce the urine to move only in a downward direction.

The above structure may be applied through various modified examples. For example, silver nanoparticles or copper oxide particles may be impregnated into the first absorbent surface in order to impart an antibacterial or deodorizing function thereto, and an asymmetric drainage pattern may be inserted into the second absorbent surface in order to induce a dispersive flow of the urine. In addition, an elastic-film-type one-way valve structure may be applied to the third absorbent surface in order to perform an automatic closing function upon upward flow. By inserting an antibacterial gel sheet into the filter layer, the passing urine may be configured to come into contact with an antibacterial substance.

As one embodiment for reinforcing the antibacterial and deodorizing functions of the first absorbent surface in this regard, silver nanoparticles or copper oxide particles may be applied onto the surface of the absorbent surface or may be mixed with the fibers inside the absorbent surface. The particles can exert a sterilizing effect from the moment of contact with the urine and can suppress bacterial proliferation even when the pad is used for a long period of time, thereby stably maintaining the sanitary condition. In addition, when a black nonwoven fabric is configured, a deodorizing function can be imparted by mixing and applying charcoal fibers, and this can be an advantageous combination for securing both antibacterial properties and deodorizing performance at the same time.

In addition, a structural feature for inducing the urine to be dispersed in a predetermined direction may be added to the second absorbent surface. For example, by forming an oblique-direction embossing pattern on the surface of the absorbent surface or by arranging the fibers in an asymmetric form, the absorbent surface can be designed to cause the urine to flow along a particular flow path within the absorbent layer. Such a configuration causes the urine to be diffused even when the urination position is repeated or concentrated, and thus can prevent supersaturation and blocking at a particular position.

In addition, a one-way valve structure using a thermoplastic resin may be inserted into the third absorbent surface. The valve may be designed so that the urine passes freely in a downward direction, but the valve is automatically closed when the urine tends to flow back upward due to pressure applied from the outside. Such a structure can prevent the urine from flowing back and wetting the surface again even when a companion animal places its body weight on the pad, and is therefore effective in physically supplementing the backflow-preventing performance.

In addition, an antibacterial gel sheet may be inserted into the spaces between the first to third absorbent surfaces. The gel sheet is disposed so as to be brought into contact during the process in which the urine passes through the inside of the filter layer, and an active antibacterial ingredient contained in the gel can dissolve into the urine, thereby suppressing the possibility of proliferation of microorganisms. In particular, the gel sheet can be fixed within the stacked structure by the gel sheet's own adhesive force even without being combined with a non-absorbent material, and can serve as a functional intermediate layer that supplements the multiple absorbent surfaces. Such a structure makes it possible to effectively maintain the sanitary condition inside the pad even when the pad is used for a long period of time.

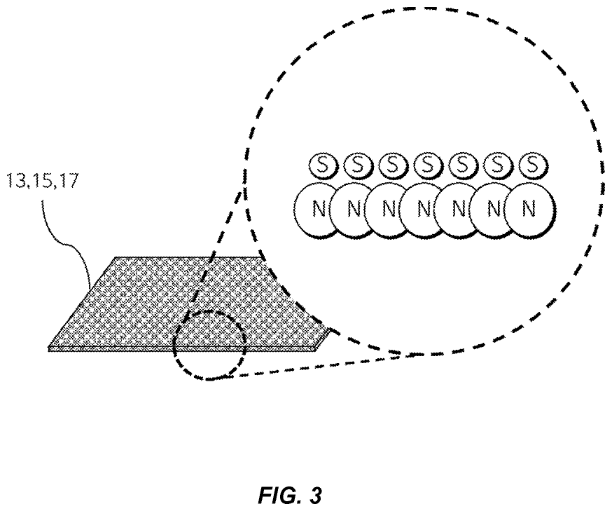

FIG. 3 shows a plan view illustrating layer-by-layer arrangement of N-type and S-type super absorbent polymer particles disposed in a first antibacterial absorbent body layer according to one embodiment of the present invention.

The first antibacterial absorbent body layer 13 is divided into an upper layer and a lower layer, and S-type super absorbent polymer particles having a fast absorption rate may be arranged at the upper portion, and N-type super absorbent polymer particles having a relatively large absorption amount may be arranged at the lower portion. Since the S-type SAP has a small average particle size and a surface activation treatment is applied thereto, the S-type SAP can immediately absorb liquid upon contact with urine. This makes it possible to implement fast surface drying so that the paw pads do not become wet even when a companion animal steps on or lies down on the corresponding portion again immediately after urinating. On the other hand, the N-type SAP arranged at the lower portion is configured of high-density particles having a large particle size and a wide internal storage volume, and can thus form a structure advantageous for storing a large amount of urine for a long period of time.

The S-type SAP and the N-type SAP are each stacked and arranged as independent layers, and may be implemented as a double-layer structure in a vertical direction rather than a conventional mixed dispersion. Such a stacked arrangement physically separates the roles of the SAP, so that a fast initial absorption function and a large-capacity storage function are performed at the same time. In addition, as shown in FIG. 3, the thickness ratio of the two layers may be set to a ratio of S-type SAP: N-type SAP=1:3 on a weight basis, and this is a value optimized in consideration of the balance between the initial absorption responsiveness and the long-term storage stability.

In order to reinforce the antibacterial function, the SAP may have an antibacterial agent based on silver ions or copper ions applied to the surface of each particle. Such antibacterially treated SAP can induce an immediate antibacterial reaction at the same time as the urine is absorbed, thereby suppressing bacterial proliferation and odor generation, and the sanitary condition can be maintained in particular even when the pad is used for a long period of time without replacement.

Owing to the structural characteristics, the first antibacterial absorbent body layer 13 may be designed so that air can circulate in the empty spaces between the SAP particles even after the urine has been absorbed into the pad, and the accumulation of internal moisture can thereby be prevented and the degradation of the performance of the SAP can be suppressed. In addition, each layer in which the SAP particles are located may be applied onto an adhesive substrate and then dried and fixed, or may be aligned and fixed inside the absorbent layer in a heat-fusion manner, so that the particles can be prevented from being skewed or moving during use.

Such a layer-by-layer SAP arrangement may be applied in the same manner not only to the first antibacterial absorbent body layer 13 but also to the second antibacterial absorbent body layer 15 and the third antibacterial absorbent body layer 17, and the functional purpose may be adjusted in accordance with the structural difference of each layer. For example, the second antibacterial absorbent body layer 15 may be configured as a single layer of only N-type SAP so as to concentrate on large-capacity storage, and the third antibacterial absorbent body layer 17 may again include S-type SAP and N-type SAP stacked therein so as to operate as an auxiliary absorbent layer for repeated absorption.

As various embodiments, the S-type SAP may be formed of a cellulose-derivative-based polymer having a fine porous structure on the surface thereof, by which the absorption reaction is fast and the liquid contact area is wide, so that immediate absorption is possible. On the other hand, the N-type SAP is a polyacrylate-based polymer that forms a cross-linked network therein so as to have a high storage capacity and maintain a stable gel state without structural deformation even in a moisture-retained state.

In addition, an intermediate support layer in which micro-perforations are formed may be inserted between the SAP particles, so that liquid can be guided to be smoothly transferred from the S-type SAP at the upper portion to the N-type SAP at the lower portion. Such an intermediate diffusion layer can physically control the movement path of the liquid, and can cause the functions of the double layer to be sequentially manifested without an absorption delay.

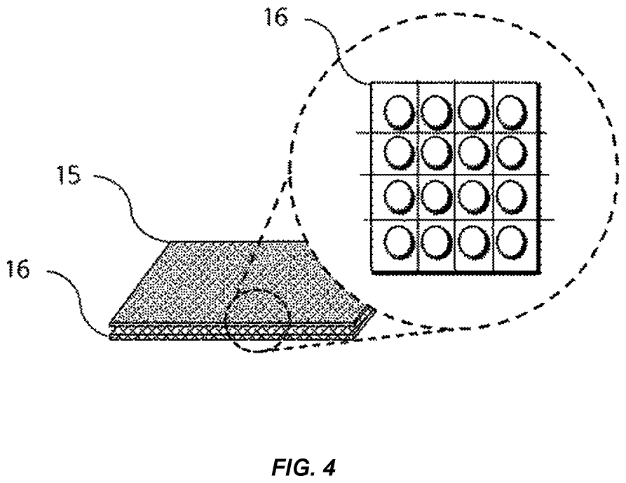

FIG. 4 shows a perspective view schematically illustrating net-structured woven fabric of a cloud sheet according to one embodiment of the present invention.

The cloud sheet 16 is a component disposed below the second antibacterial absorbent body layer 15, and can perform a role of stabilizing the arrangement of the SAP and maximizing the dispersion and absorption efficiency of the urine. As illustrated in FIG. 4, the cloud sheet 16 may be formed of a net-structured woven fabric in which fibers are cross-woven at regular intervals, and the net structure may be manufactured as a structure that maintains a constant porosity and thickness while having flexibility.

Specifically, the cloud sheet 16 may be formed by weaving a synthetic fiber such as polyester or polypropylene, and the fiber spacing may be set within an average range of 1 to 3 mm so that the SAP particles do not become detached or skewed. In addition, the SAP particles may be evenly applied and dispersed on the upper surface and the lower surface of the woven structure, and these SAP particles may be dried after application or may be dispersed and fixed on an adhesive resin. Through this, the SAP can maintain its position and stably exhibit absorption performance even under external pressure or during repeated use.

As an intermediate support layer between the absorbent body layers, the cloud sheet 16 can prevent the urine from being rapidly concentrated at a particular point and becoming saturated and can induce the urine to be uniformly dispersed over the entire area. After the urine has come into contact with the upper surface of the sheet, the urine diffuses in multiple directions through the passages between the fibers and is dispersed over a wide area before reaching the SAP, and since the cloud sheet can perform such a diffusion function, the cloud sheet can thereby be differentiated from a simple support layer of a conventional absorbent body.

In addition, since the cloud sheet can support both the upper and lower arrangements of the SAP particles, the cloud sheet can prevent the SAP from being skewed in one direction or clumped. For example, even when a companion animal repeatedly urinates on a particular portion, the cloud sheet prevents the urine from being concentrated at a particular position, thereby helping the entire surface of the SAP to operate uniformly. As a result, backflow or blocking due to supersaturation is significantly reduced, and the overall absorption performance can be maximized.

As various embodiments, the cloud sheet 16 may be formed in a single-layer structure, or may be formed in a multilayer structure in which a plurality of woven layers are stacked. For example, a composite function may be performed by combining a moisture-permeable woven fabric for inducing absorption in the upper layer and a heat-resistant woven fabric for reinforcing the SAP fixability in the lower layer. In addition, by weaving the fibers themselves with a material having an antibacterial or deodorizing function, such as silver, copper, or charcoal, mixed therein, an inherent sanitary function may be added. Furthermore, a method of further increasing the structural stability by applying an adhesive or fusion treatment to each fiber crossing point is also possible.

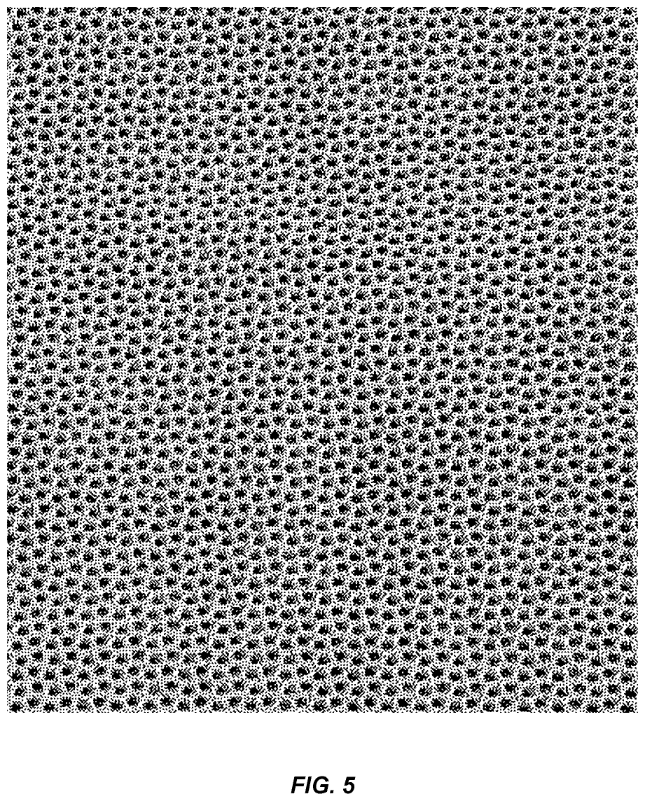

FIG. 5 is an actual photograph of a defecation pad according to one embodiment of the present invention.

The actual photograph shown in FIG. 5 is an enlarged image of the defecation pad photographed from the front, in which the structure of the upper surface that a user actually uses can be visually and clearly confirmed. In the photograph, the structure in which the top sheet layer 11 and the first filter layer 12 disposed therebelow are combined is identified with the naked eye, and the functional features of each layer are clearly shown visually.

The top sheet layer 11 is formed in a form in which fine holes of a regular pattern are arranged, and may be configured of a water-repellent material such as PE mesh or a finely perforated nonwoven fabric. The drainage hole structure can perform a role of rapidly allowing urine to pass downward, and can thereby induce the urine not to remain on the surface even when a companion animal steps on the pad again after urination. In the actual image as well, the dense and uniform perforated form of the top sheet layer is clearly shown, suggesting that the structure supports permeability and surface drying capability.

The first filter layer 12 is located below the top sheet layer 11, and in the present photograph, the filter layer is confirmed as a black background. The first filter layer 12 is configured of a triple absorbent surface structure, and the black absorbent surface at the upper portion includes a visual concealment function. In other words, even when moisture such as urine is absorbed during use, the black structure prevents the trace of the urine from being externally revealed, thereby making it possible to maintain an outwardly clean state. In the present image as well, the black color seen darkly between the drainage holes of the top sheet layer is clearly visible, which indicates that the colored first absorbent surface of the first filter layer 12 is implemented.

Additional embodiments that may be expanded in the present invention are now described.

According to one embodiment, the bottom sheet layer may be implemented not as a single PE film but as a film of a multilayer structure that also has a moisture-permeable and waterproof function. For example, the outer layer may be formed of a polypropylene film having water resistance, and the inner layer may be configured of a microporous film capable of selectively passing only water vapor or air. Such a configuration can prevent the external leakage of urine. Such a structure can be effective in preventing discomfort caused by internal moisture and degradation of the SAP performance when the pad is used for a long period of time.

In addition, a color-change indicator may be included on the outer surface of the bottom sheet layer or between the stacked internal structures. The indicator may include one or more band-shaped components disposed between a first particular layer and a second particular layer, and the band may be dyed with an indicator the color of which changes in accordance with a pH change or an increase in moisture concentration when the band comes into contact with urine.

A part of the band may be disposed on the inside in contact with the absorbent layer of the defecation pad so as to be directly brought into contact with urine during the process in which the urine is absorbed, and a part thereof may be exposed at a position on the outside of the bottom sheet layer or at a position visually identifiable from the outside. Through this, a user can intuitively determine the replacement time merely from the color change of the band exposed on the outside, without opening the pad or looking at the inside of the pad.

For example, a band that initially exhibits a yellow color may be set so as to change to a green or blue color when the pH rises or the moisture content increases due to contact with urine. The band may be disposed along an edge or a lower center line of the defecation pad, and the width of the band may be formed to be approximately 3 to 10 mm and the length thereof to be approximately 50 mm or more, so as to be configured to facilitate visual recognition.

Such an indicator band structure allows a guardian of a companion animal or a user to easily recognize the replacement timing, and can thereby improve the sanitary property and the management convenience of the product. In particular, even in an environment in which a user is out for a long time or cares for an aged dog or a companion animal having difficulty in controlling urination, the replacement time according to a change in the amount of urine can be visually indicated, so that the practicality and the distinctiveness of the product can be secured at the same time.

In addition, according to one embodiment, the present invention may be configured to be usable also as a diaper for a female companion dog, in addition to being used as a defecation pad. The defecation pad according to the present embodiment is formed in a general rectangular planar structure, and is designed so as to be capable of maintaining the same planar shape as a conventional defecation pad when the defecation pad is spread out on the floor. This is a structure for allowing a user to use the pad as a general defecation pad and, at the same time, for enabling the pad to be converted into a diaper for a companion animal through simple folding or wearing as necessary.

Specifically, an oval-shaped or droplet-shaped absorption center region corresponding to the position of the genitals of a companion dog is set at the central portion of the defecation pad, and folding lines or foldable wing portions may be formed on both sides with respect to the center region. The wing portions form a part of the rectangular pad when spread out, but can serve as flaps that wrap around both sides of the abdomen of the companion dog when the defecation pad is worn. A rubber band or hook-and-loop fastener may be attached to the wing portions, and through this, the wearing position can be flexibly fixed in accordance with the body type of the companion dog.

A tail-passage hole may be formed at one end of the defecation pad so that the tail of the companion dog can be naturally exposed. The portion at which the hole is located is positioned at the rear center when the defecation pad is worn, but in the spread-out state, the portion is integrated within the absorbent layer as a part of the rectangular structure, so that the functional structure is maintained. The periphery of the tail hole may be finished with a soft-edge-treated nonwoven fabric or a cushioning material in order to minimize skin irritation.

In addition, the entire multilayer structure is designed so that the absorption performance is not biased during wearing. The first antibacterial absorbent body layer, the second antibacterial absorbent body layer, the cloud sheet, the third antibacterial absorbent body layer, and the filter layer are stacked equally with respect to the central absorption region, and a predetermined cut line or thin pressed line may be formed at the folding lines or the folded portions so as to flexibly deform when the defecation pad is worn, while maintaining the continuity of the absorbent layer.

Through such a structure, a user can spread the defecation pad on the floor in a rectangular form and use the defecation pad like a general pad, and can convert the defecation pad into a wearable diaper by folding the wing portions and fixing them to the body of the companion dog as necessary. In particular, this structure causes the absorption performance as a defecation pad to be maintained identically even after the diaper has been worn, so that the multipurpose usability of the product can be maximized. In addition, owing to the characteristic of maintaining a rectangular shape, the product can be processed in the same manner as a general pad product group during packaging, storage, and transportation, and is therefore efficient also in terms of manufacturing and logistics.

In addition, the defecation pad of the present invention can be used as a moisture-removing pad for removing moisture adhering to shoes in order to manage the hygiene of a vehicle interior on a rainy day. In other words, the defecation pad of the present invention can also be used as a moisture-removing pad for effectively removing moisture adhering to the soles of shoes in order to manage the hygiene of a vehicle interior on a rainy day. In this case, the multilayer structure used in the defecation pad 10 of the present invention can be applied to the moisture-removing pad as it is.

In the moisture-removing pad, a dense microfiber nonwoven fabric may be applied to the top sheet layer 11 in order to immediately absorb water droplets flowing in from the soles of shoes and smoothly transfer them downward. In addition, a fine uneven pattern may be formed on the surface of the microfiber nonwoven fabric so as to widen the contact area with the shoes and to allow the water droplets to spread rapidly.

In the moisture-removing pad according to the present embodiment, since a larger load is applied when a person steps on the shoes than in the case of a defecation pad for a companion animal, there is a concern that the first to third filter layers 12, 14, and 18 may be compressed and that the moisture that has flowed into the absorbent layers may flow back to the upper portion.

In order to solve this, in the present embodiment, a polyurethane foam layer having a thickness of 2 mm and a compressive strength of 30 kPa may be inserted between the first filter layer 12 and the third filter layer 18. The foam layer is temporarily compressed when a person steps thereon and is immediately restored upon release of the load, and can thereby prevent the backflow of moisture by effectively blocking the pores of the filter layers.

In addition, a polyethylene fiber support layer having a diameter of 50 μm may be added on the upper side of the cloud sheet 16. The support layer disperses the load of a person stepping thereon over a wide area, and can thereby alleviate local compression of the filter layers and maintain a uniform pressure distribution over the entire area.

Meanwhile, the second filter layer 14 may be replaced with a porous polypropylene mesh having an average pore size of 100 μm. In the mesh, a part of the pores is closed even under the high load of a person stepping thereon, so that the backflow-preventing effect can be maximized.

By means of such improvements, the moisture-removing pad can stably suppress the backflow of moisture even under the walking load of a person, and can maintain the surface drying capability even after long-term use.

In addition, the multilayer structure of the moisture-removing pad according to the present embodiment can also be manufactured in the form of an insole that can be inserted into the inside of a shoe. In this case, the insole-type moisture-removing pad may be formed by sequentially combining the above-described first filter layer 12, first antibacterial absorbent body layer 13, second filter layer 14, second antibacterial absorbent body layer 15, cloud sheet 16, third antibacterial absorbent body layer 17, and third filter layer 18. In particular, the insole-type moisture-removing pad may be optimized by adjusting the thickness and the compressive strength of each layer so as to effectively absorb and store the moisture flowing in from the soles of shoes even in the limited space inside a shoe, and to maintain restoring force even under the repeated load that occurs during wearing. In addition, a breathable nonwoven fabric finishing layer may be added to the upper surface of the insole-type moisture-removing pad and a PE film finishing layer may be added to the lower surface thereof so as to further improve the comfort and durability for a wearer.

Starting from the basic configuration of the invention, these various embodiments can be selectively combined in accordance with functionality and user demand, and therefore the various embodiments provide a basis for broadly setting the scope of protection of the present invention and maximizing the industrial applicability.

As described above, various modifications to the embodiments of the present invention will be apparent to a person of ordinary skill in the art to which the present disclosure pertains, and the general principles defined herein may be applied to other embodiments without departing from the scope of the present disclosure. Therefore, the present invention is not limited to the embodiments presented herein, but is to be construed within the broadest scope consistent with the principles and novel features presented herein.

### REFERENCE NUMERALS

10: defecation pad
11: top sheet layer
12: first filter layer (bamboo charcoal filter)
13: first antibacterial absorbent body layer
14: second filter layer (first permeation control filter)
15: second antibacterial absorbent body layer
16: cloud sheet
17: third antibacterial absorbent body layer
18: third filter layer (second permeation control filter)
19: bottom sheet layer

1. A defecation pad (10) configured by an ordered combination of a plurality of layers, wherein the plurality of layers comprise:
a first filter layer (12);
a first antibacterial absorbent body layer (13) onto which super absorbent polymer particles are applied for absorption/storage of urine;
a second filter layer (14);
a second antibacterial absorbent body layer (15) onto which super absorbent polymer particles are applied for the absorption/storage of the urine;
a cloud sheet (16) formed of a woven fabric having a net structure for aligning particles in contact with an upper portion and a lower portion so as to be uniformly distributed;
a third antibacterial absorbent body layer (17) onto which super absorbent polymer particles are applied for the absorption/storage of the urine; and
a third filter layer (18).

2. The defecation pad (10) of claim 1, wherein the plurality of layers further include, at an uppermost end, a water-repellent top sheet layer (11) in which drainage holes for rapidly allowing the urine to permeate are arranged.

3. The defecation pad (10) of claim 2, wherein the top sheet layer (11) is formed of at least one of: polyethylene (PE) mesh, a nonwoven fabric, and Korean paper (hanji).

4. The defecation pad (10) of claim 1, wherein the plurality of layers further include, at a lowermost end, a waterproof bottom sheet layer (19) which increases a frictional force with a ground surface and prevents an outflow of the urine.

5. The defecation pad (10) of claim 1, wherein the first filter layer (12) includes:
a black first absorbent surface for concealing marks of the urine;
a second absorbent surface having a higher liquid absorption resistance than the first absorbent surface; and
a third absorbent surface having a higher liquid absorption resistance than the second absorbent surface.

6. The defecation pad (10) of claim 1, wherein, in at least one of the first antibacterial absorbent body layer (13), the second antibacterial absorbent body layer (15), and the third antibacterial absorbent body layer (17), S-type super absorbent polymer particles having a faster absorption rate than N-type super absorbent polymer particles are arranged at an upper portion, and the N-type super absorbent polymer particles having a larger absorption amount than the S-type super absorbent polymer particles are arranged at a lower portion.

7. The defecation pad (10) of claim 1, wherein, in the first antibacterial absorbent body layer (13), S-type super absorbent polymer particles having a faster absorption rate than N-type super absorbent polymer particles are arranged at an upper portion, and the N-type super absorbent polymer particles having a larger absorption amount than the S-type super absorbent polymer particles are arranged at a lower portion,
wherein, in the second antibacterial absorbent body layer (15), only the N-type super absorbent polymer particles are arranged, and
wherein, in the third antibacterial absorbent body layer (17), the S-type super absorbent polymer particles are arranged at an upper portion, and the N-type super absorbent polymer particles are arranged at a lower portion.

8. The defecation pad (10) of claim 6, wherein, for the N-type super absorbent polymer particles and the S-type super absorbent polymer particles of each of the first antibacterial absorbent body layer (13), and the third antibacterial absorbent body layer (17), a proportion of the N-type super absorbent polymer particles is higher when the defecation pad is used for a large-breed dog than when the defecation pad is used for a small-breed dog.

9. The defecation pad (10) of claim 7, wherein, for the N-type super absorbent polymer particles and the S-type super absorbent polymer particles of each of the first antibacterial absorbent body layer (13), and the third antibacterial absorbent body layer (17), a proportion of the N-type super absorbent polymer particles is higher when the defecation pad is used for a large-breed dog than when the defecation pad is used for a small-breed dog.

10. The defecation pad (10) of claim 1, wherein, in the first antibacterial absorbent body layer (13), the second antibacterial absorbent body layer (15), and the third antibacterial absorbent body layer (17), an antibacterial agent based on silver ions or copper ions is applied to surfaces of the super absorbent polymer particles.
